(19) United States
(12) Patent Application Publication
(10) Pub. No.: US 2004/0211414 A1
(43) Pub. Date: Oct. 28, 2004

(54) OXYGEN CONCENTRATION SYSTEM HAVING SELECTABLE BEDS

(75) Inventors: Dean A. CANTRILL, Bettendorf, IA (US); Michael ZUGMAIER, Bettendorf, IA (US)

Correspondence Address: MARSTELLER & ASSOCIATES, P.C., PO BOX 803302, DALLAS, TX, 75380-3302, US

(73) Assignee: LITTON SYSTEMS, INC., Los Angeles, CA (US)

(21) Appl. No.: 10/249,636

(22) Filed: Apr. 28, 2003

(51) Int. Cl. A61M 15/00

(52) U.S. Cl. 128/202.26

(57) ABSTRACT

A patient ventilator oxygen concentration system (S) has an input air supply (10) for supplying an input gas (12) at a desired pressure. An oxygen concentrating system (14) produces an oxygen concentrated gas output (16) and includes at least 2 operable molecular sieve bed modules (18). An input flow line (22) communicates input gas from the input air supply (10) to the molecular sieve bed modules (18). A medical grade air system (24) produces a medical grade air output (26). The oxygen concentrating system (14) and the medical grade air system (24) are connected in a parallel flow path of the input gas from the input air supply (10). A switch unit system (V) is in an input flow path (22) of the oxygen concentrating system (14) for controllably curtailing a portion of the input gas supplied by the input air supply (10) to at least one molecular sieve bed module (18) while having no input gas reduction effect on remaining molecular sieve bed modules. The switch unit system (V) controllably increases available gas for conversion into medical grade air.

### BACKGROUND OF INVENTION

1. Technical Field.

The present invention relates generally to oxygen concentration systems, and more particularly, to a patient ventilator oxygen concentration system using an existing source of compressed air allowing medical grade air to be simultaneously supplied along with oxygen gas from an oxygen concentrator at varying ratios.

2. Background Art.

There are a wide variety of medical applications in which oxygen and medical grade air are required. Oxygen is used for a number of respiratory care treatments. Medical grade air (as defined by United States Pharmacopia (USP) XXI) also has a number of respiratory care treatment applications. In addition to the critical care and the therapeutic benefits of these two gases, oxygen and medical grade air are used to power a range of pneumatic driven medical devices.

Hospitals have a need for oxygen and medical grade air. In military hospitals and in Europe, these needs are usually met by using oxygen concentrators for patients requiring oxygen gas and a filtration system for providing medical grade air for respiratory care treatment such as ventilators. Most United States hospitals use high-pressure gas systems or liquid oxygen to gaseous oxygen conversion systems to provide medical grade oxygen.

Hospitals use sources of compressed air. Conventional pressure swing absorption systems require a source of compressed air. Most conventional pressure swing adsorption systems use a compressor as the source of compressed air. A need exists for a system and method which can use a portion of the compressed air supply for a pressure swing absorption system, yet simultaneously allows some of the compressed air supply to be used as medical grade air.

U.S. Pat. No. 6,394,089 teaches a patient ventilator oxygen concentration system having a single switch in the air flow line between the input air supply and the plurality of molecular sieve beds in the oxygen concentrating system.

Also, U.S. Army Technical Manual TM8-3655-222-10 describes a gas generating and distribution system that produces medical grade oxygen and dry air for medical and dental tool uses. The dry air subsystem draws air from a line tap in the feed-air sub system of the gas generating and distribution system.

Numerous U.S. patents, such as U.S. Pat. Nos. 5,766,310, 5,858,063 and 6,063,169 as examples, teach oxygen concentrating systems using molecular sieve beds units having two or more molecular sieve beds comprising a molecular sieve oxygen generator. The disclosures of which are hereby incorporated in their entirety as if fully set out herein.

While the above cited references introduce and disclose a number of noteworthy advances and technological improvements within the art, none completely fulfills the specific objectives achieved by this invention.

### SUMMARY OF INVENTION

In accordance with the present invention, a patient ventilator oxygen concentration system has an input air supply for supplying an input gas at a desired pressure. An oxygen concentrating system produces an oxygen concentrated gas output and includes at least 2 operable molecular sieve bed modules. An input flow path communicates input gas from the input air supply to the molecular sieve bed modules. A medical grade air system produces a medical grade air output. The oxygen concentrating system and the medical grade air system are connected in a parallel flow path of the input gas from the input air supply. A switch unit system is in an input flow path of the oxygen concentrating system for controllably curtailing the input gas supplied by the input air supply to at least one molecular sieve bed module while having no input gas reduction effect on remaining molecular sieve bed modules. The switch unit system controllably increases available gas for conversion into medical grade air.

It is, therefore, an object of the present invention to provide a patient ventilator oxygen concentration system using an existing source of compressed air to provide oxygen gas and medical grade air.

Another object of the present invention is to provide a patient ventilator oxygen concentration system which simultaneously provides medical grade air from an air filtration system supplied along with oxygen gas from an oxygen concentration system.

Yet another object of the present invention is to produce both therapeutic oxygen and medical grade air flow quantities and at specific pressures compatible with patient ventilation devices.

It is another object of the present invention to provide a patient ventilator oxygen system which can maintain oxygen purity by using a modular bed design.

It is another object of the present invention to provide a patient ventilator oxygen system using multiple sets of zeolite beds.

The present invention called a patient ventilator oxygen concentration system advantageously utilizes an existing suitable air supply and provides a modular oxygen concentrator that uses the existing air supply and a medical grade air filtration package for providing medical grade using the existing air supply. The oxygen concentrator has multiple bed pairs which can be selectively activated. If one of the multiple bed pairs is not activated, the excess air provided by the existing air supply is filtered and medical grade air is supplied instead of oxygen gas for use with patient ventilators. Advantageously, the present invention obtains a large increase in medical grade air flow at the expense of very little oxygen flow while maintaining oxygen purity using the existing air supply. It is not possible to make this air/oxygen trade-off using a conventional pressure swing absorption (PSA) system for maintaining oxygen purity not having multiple bed pairs. The present invention provides an oxygen concentrator having a pneumatic circuit using a modular bed design. Each bed pair may use approximately three Standard Cubic Feet per Minute (SCFM) (80 SLPM) to produce five Standard Liters Per Minute (SLPM) of oxygen. Shutting down at least one selected bed pair reduces the oxygen flow but increases the available compressed air to be converted into medical grade air. The remaining bed pair maintains their oxygen purity because the compressed air supply is not reduced. By contrast, if a single bed pair system were used, reducing the oxygen output would not free up a significant amount of feed air to be converted into medical grade air. Using a conventional oxygen concentrator, if the demand for medical grade air increased beyond rated flow, the oxygen purity would decrease due to the transfer of feed air from the PSA bed pair to the medical grade air system.

These and other objects, advantages and features of this invention will be apparent from the following description taken with reference to the accompanying drawings, wherein is shown the preferred embodiments of the invention.

### BRIEF DESCRIPTION OF DRAWINGS

A more particular description of the invention briefly summarized above is available from the exemplary embodiments illustrated in the drawing and discussed in further detail below. Through this reference, it can be seen how the above cited features, as well as others that will become apparent, are obtained and can be understood in detail. The drawings nevertheless illustrate only typical, preferred embodiments of the invention and are not to be considered limiting of its scope as the invention may admit to other equally effective embodiments.

FIG. 1 is a pneumatic circuit according to the present invention.

### DETAILED DESCRIPTION

So that the manner in which the above recited features, advantages, and objects of the present invention are attained can be understood in detail, more particular description of the invention, briefly summarized above, may be had by reference to the embodiment thereof that is illustrated in the appended drawings. In all the drawings, identical numbers represent the same elements.

A patient ventilator oxygen concentration system S has an input air supply 10 for supplying an input gas 12 at a desired pressure. An oxygen concentrating system 14 produces an oxygen concentrated gas output 16 and includes at least 2 operable molecular sieve bed modules 18. An input flow path or line 22 communicates input gas from the input air supply 10 to the molecular sieve bed modules 20a or 20b. A medical grade air system 24 produces a medical grade air output 26. The oxygen concentrating system 14 and the medical grade air system 24 are connected in a parallel flow path of the input gas from the input air supply 10. A switch unit system V is in an input flow path 22 of the oxygen concentrating system 16 for controllably curtailing or blocking the input gas supplied by the input air supply 10 to at least one molecular sieve bed module 18 while having no input gas reduction effect on remaining molecular sieve bed modules. The switch unit system V controllably increases available gas for conversion into medical grade air.

The primary operation of the ventilator system S is to provide a selected amount or flow rate of medical grade air at a desired pressure while simultaneously providing a selected amount or flow rate of concentrated oxygen at a desired pressure. Higher flow rates of medical grade air are achieved by controlling the number of operable molecular sieve bed modules 18. Being able to selectively shut down or effectively disconnect or block a molecular sieve bed module 20a or 20b results in an increase in the proportion of medical grade air generated by the ventilator system S relative to concentrated oxygen, or equivalently, a reduction in the relative proportion of concentrated oxygen with regard to the medical grade air output flow rate.

Shutting down or blocking additional molecular sieve bed units 18 further adjusts the proportion of medical grade air and concentrated oxygen by increasing the flow rate of the medical grade air and reducing the flow rate of the concentrated oxygen.

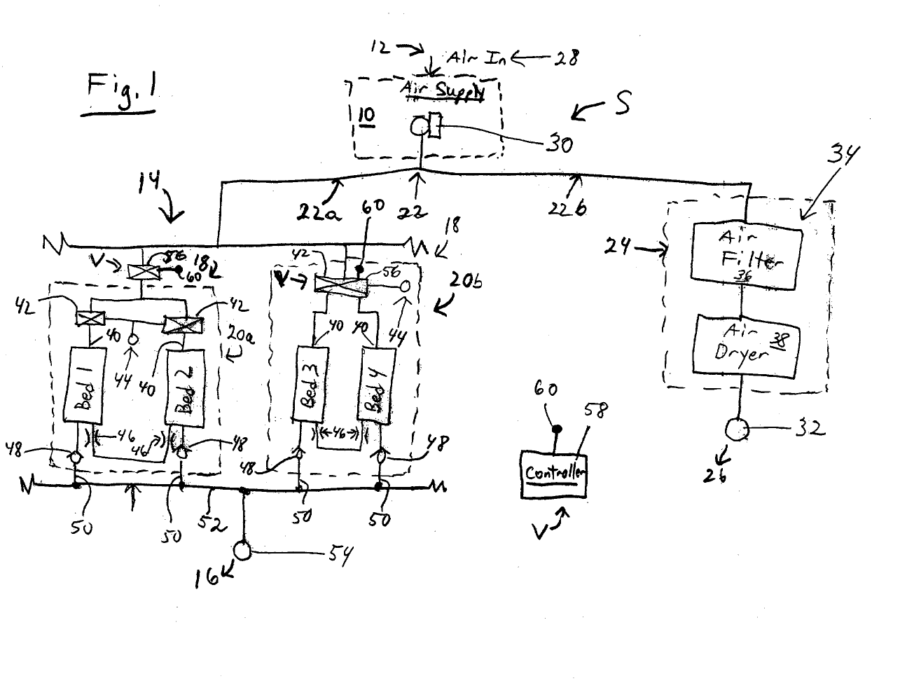

Specifically referring to FIG. 1, ambient air or other input gas 12 enters the input air supply unit 10 through air input 28. An air compressor 30, such as a scroll compressor or other suitable type, provides compressed air at specific flow and pressure values to support the medical grade air package 24 and oxygen concentrator

An input flow path 22 conveys the pressurized air from the air supply 10 to the medical grade air package 24 and oxygen concentrator 14 through parallel flow branches 22a and 22b. Such flow path 22 typically is a pipe, tube or other known pneumatic means adapted to convey the pressurized air without significant loss. Preferably, the input gas from the air supply 10 is distributed between only the oxygen concentrating system 14 and the medical grade air system 24.

The medical grade air system 24 generally processes the compressed input air into a form suitable for medical purposes, such as patient ventilation, breathing, medical instrument operation, such a surgical canullas, or the like. Air tanks may optionally be filled by adding another air compressor to the medical grade output or connection 32.

Typically, the medical grade air system 24 includes an air cleaning system 34 including a known type of air filter unit 36 and an air dryer unit 38, as are well known in the art. Passing the initially compressed input gas through the medical grade air circuit 24 results in an output 26 of air having the characteristics of purity and humidity as is appropriate for the intended use of the medical grade output.

Optionally, pressure regulators, exhaust valves, air\water separators or the like may be connected to or form the output 32 from the medical grade air unit 24 or at any other location in the pneumatic circuitry chosen.

The concentrating system 14 includes at least two molecular sieve bed units or modules 18 connected in a parallel pneumatic flow path. Two molecular sieve bed modules 20a and 20b are shown by way of example in FIG. 1, although any number greater than two may be chosen.

Each molecular sieve bed module 18 preferably includes at least two individual molecular sieve beds bed 1 and bed 2 or bed 3 and bed 4 also connected in a pneumatic parallel flow path within the molecular sieve bed module 18.

Typically, the molecular sieve beds bed 1, bed 2, bed 3, and bed 4 are known zeolite beds with each having an inlet 40.

Each molecular sieve bed bed 1, bed 2, bed 3, bed 4 has a sequencing valve 42 in the input flow path to sequentially port the air to the appropriate sieve bed, either the bed 1 or bed 2 of unit 20a, or bed 3 or bed 4 of unit 20b. Such valve 42 may be a known slide valve, rotary valve or other suitable type. The two oxygen beds, bed 1 and bed 2, or bed 3 and bed 4, operate as an alternating pair so that when one bed is pressurized, adsorbing nitrogen, and producing oxygen-enriched product gas, the other bed is vented to ambient air using port 44. Also, schematically shown are cross flow orifices 46, check valves 48, and output tubing 50. The desired product gas, generally concentrated oxygen, flows into manifold 52 and is drawn out through output gas junction 54. Similarly, the oxygen gas junction 54 may be attached to optional pressure regulators, valves, or the like as desired.

Each molecular sieve bed module 18 has a switch system V including a controllable shut-off valve 56 mounted in the input air flow path 22 between the air supply 10 and at least one of the molecular sieve bed modules 18. Valve 56 operates at a minimum in a manner to either fully pass or block air flow into the selected molecular sieve bed module 18 or at any other intermediate state that may be desired for the specific arrangement designed. The shut-off valve 56 effectively disconnects the appropriate molecular sieve bed module 18 from the input gas supply 10.

For molecular sieve bed module 20a the shut-off valve V is a separate unit and is mounted between the input flow path 22 and valves 42. However, for molecular sieve bed module 20b, the shut-off valve 56 is incorporated into the sequencing valve 42.

Optionally, an electronic or mechanical switch controller 58 mates with one or more shut-off valves 56 forming the controllable switching system V to remotely operate the desired shut-off valve 56. FIG. 1 schematically shows controller 58 connected to the valves 56 with operable connections 60. The controller 58 may cause a solenoid to activate the valve, as is commonly known in the art. Preferably, the controller 58 would be correlated to shutting-off or blocking one or more molecular sieve be modules 18 as desired to achieve the desired flow rate or proportion of medical grade air to oxygen.

Yet another alternative embodiment would place the blocking valves in the discharge or output lines 50 exiting the molecular sieve beds. Such alternative placement of blocking valves would also act to shut down one or more individual sieve beds or molecular sieve bed units by preventing the passage of the gas through the molecular sieve beds.

The present invention simultaneously provides oxygen gas and medical grade air using a source of compressed air 10 providing a volume of compressed air. One or more molecular sieve bed units or modules 18 is selected to receive a portion of the volume of compressed air while, at the same time, the selection also causes the flow of the compressed air to the remaining operable molecular sieve bed units 18 to be impeded or blocked.

A portion of the volume of compressed air flows through the selected one or more molecular sieve bed units 18 and results in an output oxygen gas exiting from the selected molecular sieve bed units. The remainder of the volume of the compressed air flows into an air cleaning system 24 and then to one or more air outlets 32.

The foregoing disclosure and description of the invention are illustrative and explanatory thereof, and various changes in the size, shape and materials, as well as in the details of the illustrated construction may be made without departing from the spirit of the invention.

1. A patient ventilator oxygen concentration system, comprising:
an input air supply for supplying an input gas at a desired pressure;
an oxygen concentrating system for producing oxygen concentrated gas output including:
at least 2 operable molecular sieve bed modules; and,
an input flow path for communicating input gas from
the input air supply to the molecular sieve bed modules;
a medical grade air system for producing a medical grade air output;
the oxygen concentrating system and the medical grade air system connected in a parallel flow path of the input gas from the input air supply; and,
a switch unit system in an input flow path of the oxygen concentrating system for controllably curtailing a portion of the input gas supplied by the input air supply to at least one molecular sieve bed module while having no input gas reduction effect on remaining molecular sieve bed modules;
whereby the switch unit system controllably increases available gas for conversion into medical grade air.

2. The invention of claim wherein selected activation of the switch unit causes a reduction in the number of operable molecular sieve bed modules.

3. The invention of claim wherein reducing the number of operable molecular sieve bed modules causes an increase of gas available for conversion into medical grade air.

4. The invention of claim wherein selectively reducing a number of operable molecular sieve bed modules causes an increase of gas available for conversion into medical grade air.

5. The invention of claim wherein controlled activation of the switch unit causes one or more operable molecular sieve bed modules to be turned off.

6. The invention of claim wherein the input gas from the input air supply is distributed between only the oxygen concentrating system and the medical grade air system.

7. The invention of claim wherein the switch unit system further includes at least one controllable valve connected in an air path between the input air supply and a molecular sieve bed module to controllably disconnect the molecular sieve bed module from the input gas supply.

8. The invention of claim wherein the input air supply further includes an air compressor.

9. The invention of claim wherein the air compressor is a scroll compressor.

10. The invention of claim wherein each of the molecular sieve bed modules includes at least 2 individual molecular sieve beds.

11. The invention of claim wherein the molecular sieve beds are zeolite beds with each having an inlet.

12. The invention of claim wherein the medical grade air system further includes an air cleaning system and at least one medical grade air output connected to the air cleaning system.

13. The invention of claim wherein the air cleaning system includes an air filter.

14. The invention of claim wherein the air cleaning system includes an air dryer.

15. A method of simultaneously providing oxygen gas and medical grade air using a source of compressed air providing a volume of compressed air, comprising:
selecting one or more molecular sieve bed units to receive a portion of the volume of compressed air and causing the flow of the compressed air to remaining operable molecular sieve bed units to be impeded;
flowing a portion of the volume of compressed air through the selected one or more molecular sieve bed units and providing an output oxygen gas therefrom; and,
flowing the remainder of the volume of the compressed air into an air cleaning system and then to air outlets.

16. The method of claim wherein the air cleaning system includes an air filter.

17. The method of claim wherein the air cleaning system includes an air dryer.
